(19) United States
(12) Patent Application Publication
(10) Pub. No.: US 2024/0193325 A1
(43) Pub. Date: Jun. 13, 2024

(54) MOVEMENT ANALYSIS SYSTEM, MOVEMENT ANALYSIS METHOD, AND PROGRAM

(71) Applicant: TOYOTA JIDOSHA KABUSHIKI KAISHA, Toyota-shi (JP)

(72) Inventors: Eisuke AOKI, Toyota-shi (JP); Tadashi ODASHIMA, Seto-shi (JP); Yuko NAKAHIRA, Nagakute-shi (JP); Hidekazu NISHIGAKI, Nagakute-shi (JP)

(73) Assignee: TOYOTA JIDOSHA KABUSHIKI KAISHA, Toyota-shi (JP)

(21) Appl. No.: 18/383,785

(22) Filed: Oct. 25, 2023

(30) Foreign Application Priority Data
Dec. 8, 2022 (JP) 2022-196179

(51) Int. Cl. G06F 30/23; A61B 5/11

(52) U.S. Cl. CPC G06F 30/23; A61B 5/1107

(57) ABSTRACT

A method for training a specific muscle is provided to a user. A movement analysis system includes a muscle movement unit configured to increase a muscle output of only a muscle that a user wants to move and move only the muscle on a human body model of a finite element simulator, a position information calculation unit configured to calculate, as time-series position information, a trajectory in which each joint is moved when the muscle is moved by the muscle movement unit on the finite element simulator, and a movement provision unit configured to provide movements of the user for reproducing the time-series trajectory of each joint based on the position information calculated by the position information calculation unit.

### CROSS REFERENCE TO RELATED APPLICATIONS

This application is based upon and claims the benefit of priority from Japanese patent application No. 2022-196179, filed on Dec. 8, 2022, the disclosure of which is incorporated herein in its entirety by reference.

### BACKGROUND

The present disclosure relates to a movement analysis system, a movement analysis method, and a program for analyzing movements of a person.

A muscle condition estimation apparatus for constructing an initial user model by reflecting user parameters in a general human body model based on skeletal data and muscle data is known (see, for example, Japanese Patent No. 6997228).

### SUMMARY

However, although the muscle condition estimation apparatus constructs an initial user model, it does not provide a user with a method for training a specific muscle. Further, an apparatus using machine learning or the like also does not provide a user with this method.

The present disclosure has been made to solve such a problem, and a main object thereof is to provide a movement analysis system, a movement analysis method, and a program for providing a user with a method for training a specific muscle.

An example aspect of the present disclosure in order to achieve the above object is a movement analysis system including:
a muscle movement unit configured to increase a muscle output of only a muscle that a user wants to move and move only the muscle on a human body model of a finite element simulator;
a position information calculation unit configured to calculate, as time-series position information, a trajectory in which each joint is moved when the muscle is moved by the muscle movement unit on the finite element simulator; and
a movement provision unit configured to provide movements of the user for reproducing the time-series trajectory of each joint based on the position information calculated by the position information calculation unit.

In this example aspect, the movement provision unit may include a display unit configured to display to the user the movements of the user for reproducing the time-series trajectory of each joint.

In this example aspect, the movement provision unit may include an orthosis or a teaching robot configured to assist the user in performing the movements for reproducing the time-series trajectory of each joint.

In this example aspect,
the finite element simulator may include:
   a muscle controller unit including:
      a target angle input unit configured to receive a target angle of each joint desired to be moved by the human body model;
      a deviation calculation unit configured to calculate a deviation of a joint angle calculated by an angle calculation unit from the target angle in the target angle input unit;
      a PID muscle control unit configured to perform PID control based on the deviation calculated by the deviation calculation unit and calculate an operation amount of each muscle for posture control;
      a muscle activity calculation unit configured to calculate a muscle activity of each muscle based on the operation amount of each muscle calculated by the PID muscle control unit; and
      the angle calculation unit configured to calculate the joint angle of each joint of the human body model based on coordinates of nodes fed back from the human body model of a muscle solid model unit; and
   the muscle solid model unit comprising a model analysis unit configured to perform finite element analysis on the human body model based on the muscle activity of each muscle calculated by the muscle activity calculation unit and move each muscle of the human body model based on a result of the finite element analysis.

Another example aspect of the present disclosure to achieve the above object is a movement analysis method including:
increasing a muscle output of only a muscle that a user wants to move and move only the muscle on a human body model of a finite element simulator;
calculating, as time-series position information, a trajectory in which each joint is moved when the muscle is moved on the finite element simulator; and
providing movements of the user for reproducing the time-series trajectory of each joint based on the position information.

Another example aspect of the present disclosure to achieve the above object is a program for causing a computer to execute:
processing of increasing a muscle output of only a muscle that a user wants to move and move only the muscle on a human body model of a finite element simulator;
processing of calculating, as time-series position information, a trajectory in which each joint is moved when the muscle is moved on the finite element simulator; and
processing of providing movements of the user for reproducing the time-series trajectory of each joint based on the position information.

According to the present disclosure, it is possible to provide a movement analysis system, a movement analysis method, and a program for providing a user with a method for training a specific muscle.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram showing a schematic system configuration of a movement analysis system according to an embodiment;

FIG. 2 is a block diagram showing a schematic system configuration of a finite element simulator according to the embodiment;

FIG. 3 is a flowchart showing a flow of a movement analysis method according to the embodiment; and

FIG. 4 shows a temporal shift of a hip joint angle when only iliopsoas muscle of a human body model in a seated position is contracted on the finite element simulator.

### DESCRIPTION OF EMBODIMENTS

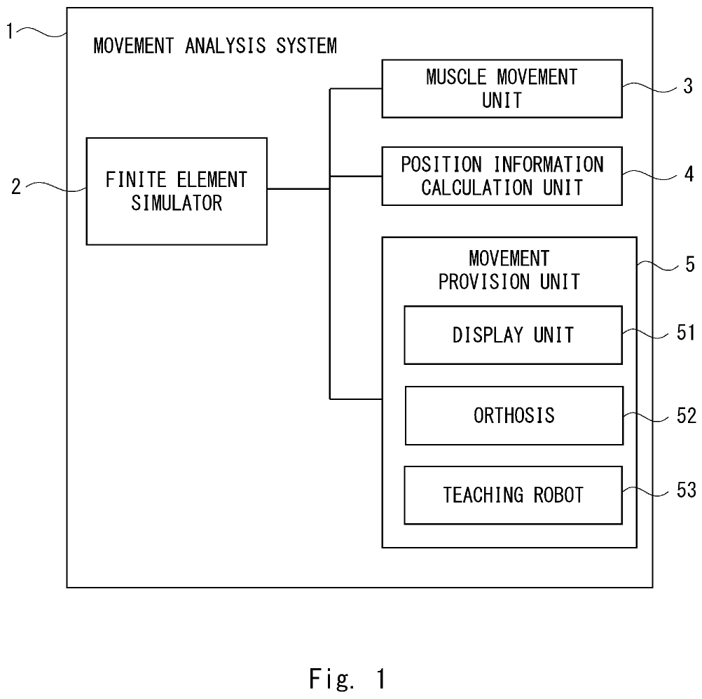

An embodiment of the present disclosure will be described below with reference to the drawings. FIG. 1 is a block diagram showing a schematic system configuration of a movement analysis system according to this embodiment. A movement analysis system 1 according to this embodiment presents a user with a method for training a specific muscle.

The movement analysis system 1 according to this embodiment includes a finite element simulator 2, a muscle movement unit 3, a position information calculation unit 4, and a movement provision unit 5.

The movement analysis system 1 includes a hardware configuration of a normal computer including, for example, a processor such as a CPU (Central Processing Unit) and a GPU (Graphics Processing Unit), an internal memory such as a RAM (Random Access Memory) and a ROM (Read Only Memory), a storage device such as an HDD (Hard Disk Drive) and an SSD (Solid State Drive), an input/output I/F for connecting a peripheral device such as a display, and a communication I/F for communicating with a device external to the apparatus.

The finite element simulator 2 according to this embodiment actively moves a human body model. Specifically, the finite element simulator 2 according to this embodiment includes a muscle controller described later which actively moves a human body model.

A human body model of related art is disclosed in Japanese Patent No. 3760793 and the like.

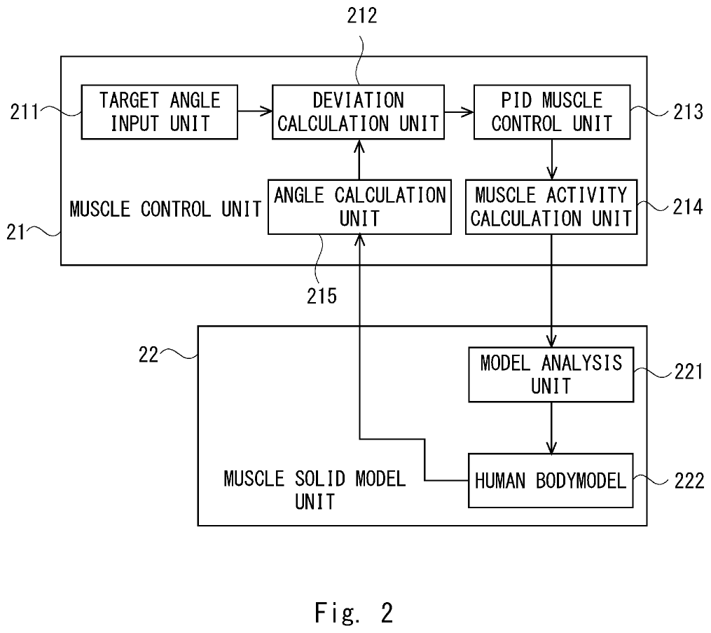

FIG. 2 is a block diagram showing a schematic system configuration of the finite element simulator according to this embodiment. The finite element simulator 2 according to this embodiment includes a muscle control unit 21 for controlling muscles of a human body model 222 and a muscle solid model unit 22.

The muscle control unit 21 includes a target angle input unit 211, a deviation calculation unit 212, a PID muscle control unit 213, a muscle activity calculation unit 214, and an angle calculation unit 215.

The temporal shift of target angles for each joint that the user wants to move on the human body model 222 is input to the target angle input unit 211. The target angle of each joint corresponding to the movement of each joint may be input to the target angle input unit 211 as a time history.

For example, in the target angle input unit 211, the knee joint is input as 180 degrees at 0 seconds, 90 degrees at 1 second, . . . and the like. The time history of the angle change from the initial angle may be input as an input value. The time history of the target angle is set according to, for example, a posture of a user (angle change of each joint) during training. The target angle input unit 211 outputs the input target angle to the deviation calculation unit 212.

The deviation calculation unit 212 calculates a deviation of the target angle output from the target angle input unit 211 and a joint angle from the angle calculation unit 215. The deviation calculation unit 212 outputs the calculated deviation to the PID muscle control unit 213.

The PID muscle control unit 213 performs PID (Proportional-Integral-Differential) control based on the deviation calculated by the deviation calculation unit 212 and calculates an operation amount of each muscle for posture control. The PID muscle control unit 213 outputs the calculated operation amount of each muscle to the muscle activity calculation unit 214.

The muscle activity calculation unit 214 calculates muscle activity of each muscle based on the operation amount of each muscle output from the PID muscle control unit 213. The muscle activity calculation unit 214 outputs the calculated muscle activity to the model analysis unit 221 of the muscle solid model unit 22.

The angle calculation unit 215 calculates the joint angle of each joint of the human body model 222 based on the coordinates of the nodes fed back from the human body model 222 of the muscle solid model unit 22 as described later. The angle calculation unit 215 outputs the calculated joint angle of each joint to the deviation calculation unit 212.

The muscle solid model unit 22 has a model analysis unit 221 and a human body model 222.

The model analysis unit 221 performs finite element analysis on the human body model 222 based on the muscle activity of each muscle output from the muscle activity calculation unit 214. The model analysis unit 221 causes each muscle of the human body model 222 to move based on a result of the finite element analysis. As described above, the human body model 222 outputs the coordinates of the nodes as a result of the movements thereof to the angle calculation unit 215 of the muscle control unit 21.

The coordinates of the nodes are, for example, three points, such as a lower node, an upper node, and a front node for each part of the human body model 222, such as the head, chest, and pelvic region. The specific node positions are shown below in the order of the lower part, the upper part, and the front part of the human body model 222.
Head: Center of gravity of head, parietal point, glabellar
Chest: 12th thoracic vertebrae, 1st thoracic vertebrae, sternal stalk
Pelvic region: Coccyx tip, sacral base, sacral promontory
Femoral region: Femoral intercondylar fossa, femoral head center, femoral patellar surface
Lower leg: Tibial talar glenoid fossa, tibial intercondylar eminence, tibial tuberosity
Foot: Calcaneal eminence medial process, talar trochlea, great toe tip
Scapular region: Lower scapula, upper scapula, upper scapula front
Humerus: Radial groove of the humerus, center of the humeral head, lesser tubercle of the humerus
Hand: Dorsal surface distal of the navicular bone, dorsal surface proximal of the navicular bone, palmar surface distal of the navicular bone.

By the way, joints such as a human knee allow bones to move by sliding against each other. However, each of the joints in human body models of the related art is composed of a single joint to reduce computational load. Also, while muscles are reproduced in human body models of the related art and ligaments are not, and thus the trajectories of the hands and feet in the human body models of the related art may differ significantly from the actual trajectories observed in real human movements.

In addition, when a human joint moves, the muscles contract and their cross-sectional area changes in the presence of friction between tissues such as muscles and ligaments. On the other hand, the sections of the body where the muscles are attached are reproduced in human body models of related art, but they do not account for changes in muscle cross-sectional areas when a plurality of muscles within the tissue undergo contraction, nor do they consider the friction that occurs between tissues. As a result, human body models of related art do not accurately estimate the muscle contraction forces involved when a person moves his/her joints.

On the other hand, the human body model 222 according to this embodiment has a skeletal structure similar to that of a person, and muscles and ligaments are attached to the skeletal structure. The strain and stress distribution of the tissue can be visualized. The contact between the muscles can also be reproduced. In the human body model 222, the changes in the cross-sectional area of the muscles when the muscles contract are reproduced, and the friction between the tissues of the muscles, skin, and ligaments during the changes are reflected in the human body model 222.

The muscle movement unit 3 increases a muscle output only for the muscle the user wants to move (hereinafter referred to as “target muscle”) on the human body model 222 of the finite element simulator 2, thereby causing movement exclusively in the target muscle. The muscle movement unit 3 may contract only the target muscle on the human body model 222 of the finite element simulator 2.

The information (gluteus maximus, iliac muscle, etc.) about the target muscle is set in the muscle movement unit 3 via, for example, an input apparatus.

For example, the muscle movement unit 3 inputs 100% constant muscle activity to the target muscles and 0% constant muscle activity to the other muscles to the human body model 222 of the finite element simulator 2. By doing so, the muscle output is increased exclusively for the target muscle, resulting in the movement of only the target muscle.

The position information calculation unit 4 calculates the trajectory in which each joint is moved when the target muscle is moved on the finite element simulator 2 as position information in a time series. The position information calculation unit 4 may calculate the trajectory in which each joint, hand, and foot is moved when the target muscle is moved as the position information in a time series. The position information calculation unit 4 outputs the calculated position information to the movement provision unit 5.

The movement provision unit 5 provides movements of the user for reproducing the time series trajectory of each joint based on the position information output from the position information calculation unit 4.

The movement provision unit 5 may have a display unit 51 for displaying the user's movements reproducing the time series trajectory of each joint to the user. The display unit 51 displays, for example, trajectories of joints, hands, feet, and other body parts. The user can perform training by moving only the target muscle while looking at the trajectories of joints, hands, feet, and other body parts displayed on the display unit 51.

In addition, the movement provision unit 5 may have an orthosis 52 or a teaching robot 53 for assisting the user's movements so as to perform a movement reproducing the time-series trajectory of each joint. The orthosis 52 is attached to the user and is configured to forcibly perform the movement following the trajectories.

Moreover, when the user wears the teaching robot 53 and the teaching robot 53 performs movements following the trajectories, the user mimics those movements. As a result, the user reproduces the time-series trajectories of each joint's movements by following the orthosis 52 or the teaching robot 53.

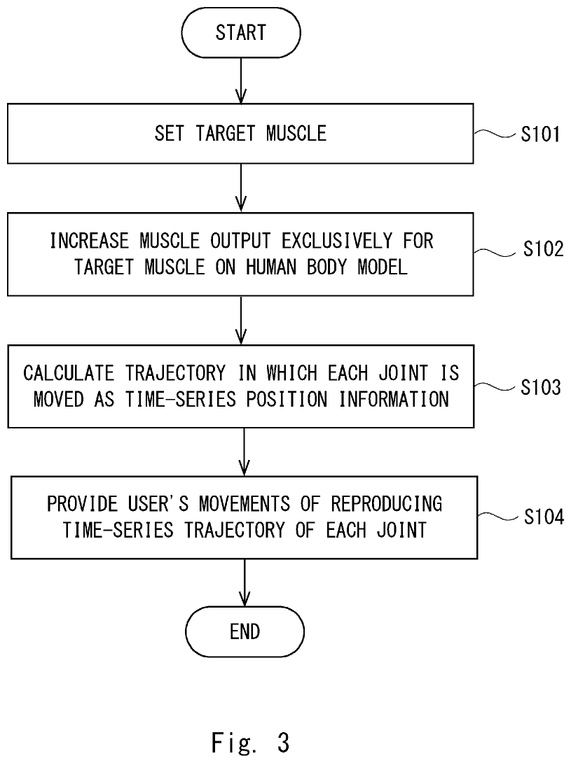

Next, a flow of the movement analysis method according to this embodiment will be described in detail. FIG. 3 is a flowchart showing a flow of the movement analysis method according to this embodiment. First, a target muscle is set in the muscle movement unit 3 (Step S101).

The human body model 222 of the finite element simulator 2 moves according to the temporal shift of the target angle of each joint input to the target angle input unit 211 or the muscle activity of the target muscle. At this time, the muscle movement unit 3 increases the muscle output exclusively for the target muscle on the human body model 222 of the finite element simulator 2, causing movement in only the target muscles (Step S102).

The position information calculation unit 4 calculates the trajectory of each joint when the target muscle is moved on the finite element simulator 2 as the position information in a time series (Step S103).

The movement provision unit 5 provides the user's movements of reproducing the time-series trajectory of each joint based on the position information calculated by the position information calculation unit 4 (Step S104).

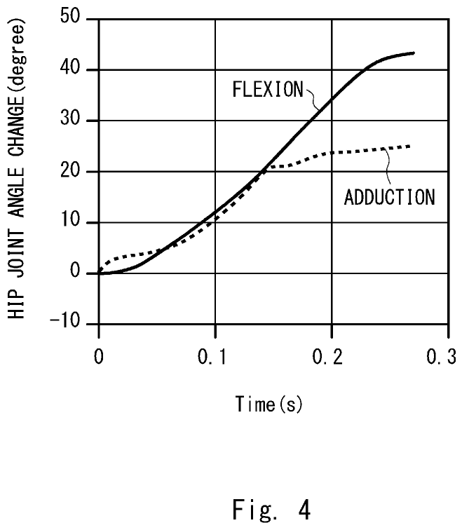

Next, a simulation has been conducted on the human body model 222 in a seated posture on the finite element simulator 2, where only the muscle output of the iliopsoas muscle (composed of the gluteus maximus and iliopsoas muscle) is increased, causing contraction exclusively in the iliopsoas muscle. FIG. 4 shows the temporal shift of the hip joint angle when only the iliopsoas muscle of the human body model 222 in the seated posture is contracted on the finite element simulator 2 as described above.

When the iliopsoas muscle of the human body model 222 is contracted on the finite element simulator 2, the hip joint angle in the flexion direction gradually increases and also the hip joint angle in the adduction direction gradually increases as shown in FIG. 4.

As a result, it can be seen that the hip joint does not just flexes straight but flexes inward. Therefore, if the user simply flexes the hip joint straight, not only the iliopsoas muscle but also other muscles will be used to perform the movement.

According to the movement analysis system 1 of this embodiment, for example, if a deep muscle such as the iliopsoas muscle is set as the target muscle in the muscle movement unit 3 as described above, the muscle movement unit 3 increases the muscle output exclusively for deep muscles on the human body model 222 of the finite element simulator 2, causing movement in only the deep muscles. The position information calculation unit 4 calculates the trajectory in which each joint is moved when the deep muscles are moved on the finite element simulator 2 as the position information in a time series.

The movement provision unit 5 provides the user's movements of reproducing the time series trajectory of each joint based on the position information calculated by the position information calculation unit 4. By simply following the movements provided by the movement provision unit 5, the user can efficiently train the deep muscles, which are particularly difficult to train.

The movement analysis system 1 according to this embodiment includes the muscle movement unit 3 configured to increase a muscle output of only a muscle that a user wants to move and move only the muscle on the human body model 222 of the finite element simulator 2, the position information calculation unit 4 configured to calculate, as time-series position information, trajectory in which each trajectory in which each joint is moved when the muscle is moved by the muscle movement unit 3 on the finite element simulator 2, and the movement provision unit 5 configured to provide movements of the user for reproducing the time-series trajectory of each joint based on the position information calculated by the position information calculation unit 4. Thus, a method for training a specific muscle can be provided to a user.

Although the embodiment of the present disclosure has been described, the embodiment is presented as an example and is not intended to limit the scope of the disclosure. These novel embodiments can be implemented in various other forms, allowing for various omissions, substitutions, and modifications within the scope of the invention without departing from its essence. These embodiments and variations thereof are encompassed within the scope and essence of the invention, as well as within the scope of the claims filed in the patent application.

For example, the process shown in FIG. 3 may be implemented by causing a processor to execute a computer program.

The program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (Read Only Memory), CD-R, CD-R/W, and semiconductor memories (such as mask ROM, PROM (Programmable ROM), EPROM (Erasable PROM), flash ROM, RAM (random access memory), etc.).

The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

The components of the movement analysis system 1 according to the embodiments described above are not only implemented by programs, but some or all of them can also be implemented by specialized hardware, such as ASICs (Application Specific Integrated Circuits) or FPGAs (Field-Programmable Gate Arrays).

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A movement analysis system comprising:
a muscle movement unit configured to increase a muscle output of only a muscle that a user wants to move and move only the muscle on a human body model of a finite element simulator;
a position information calculation unit configured to calculate, as time-series position information, a trajectory in which each joint is moved when the muscle is moved by the muscle movement unit on the finite element simulator; and
a movement provision unit configured to provide movements of the user for reproducing the time-series trajectory of each joint based on the position information calculated by the position information calculation unit.

2. The movement analysis system according to claim 1, wherein the movement provision unit includes a display unit configured to display to the user the movements of the user for reproducing the time-series trajectory of each joint.

3. The movement analysis system according to claim 1, wherein the movement provision unit includes an orthosis or a teaching robot configured to assist the user in performing the movements for reproducing the time-series trajectory of each joint.

4. The movement analysis system according to claim 1, wherein
the finite element simulator comprises:
a muscle controller unit comprising:
a target angle input unit configured to receive a target angle of each joint desired to be moved by the human body model;
a deviation calculation unit configured to calculate a deviation of a joint angle calculated by an angle calculation unit from the target angle in the target angle input unit;
a PID muscle control unit configured to perform PID control based on the deviation calculated by the deviation calculation unit and calculate an operation amount of each muscle for posture control;
a muscle activity calculation unit configured to calculate a muscle activity of each muscle based on the operation amount of each muscle calculated by the PID muscle control unit; and
the angle calculation unit configured to calculate the joint angle of each joint of the human body model based on coordinates of nodes fed back from the human body model of a muscle solid model unit; and
the muscle solid model unit comprising a model analysis unit configured to perform finite element analysis on the human body model based on the muscle activity of each muscle calculated by the muscle activity calculation unit and move each muscle of the human body model based on a result of the finite element analysis.

5. A movement analysis method comprising:
increasing a muscle output of only a muscle that a user wants to move and move only the muscle on a human body model of a finite element simulator;
calculating, as time-series position information, a trajectory in which each joint is moved when the muscle is moved on the finite element simulator; and
providing movements of the user for reproducing the time-series trajectory of each joint based on the position information.

6. A non-transitory computer readable medium storing a program for causing a computer to execute:
processing of increasing a muscle output of only a muscle that a user wants to move and move only the muscle on a human body model of a finite element simulator;
processing of calculating, as time-series position information, a trajectory in which each joint is moved when the muscle is moved on the finite element simulator; and
processing of providing movements of the user for reproducing the time-series trajectory of each joint based on the position information.
